(19) United States
(12) Patent Application Publication
(10) Pub. No.: US 2007/0292939 A1
(43) Pub. Date: Dec. 20, 2007

(54) Insert with concavity for organic culture and imaging

(76) Inventor: Stephen Liye Chen, El Monte, CA (US)

Correspondence Address: Stephen Liye Chen, 12331 Felipe Street, El Monte, CA, 91732, US

(21) Appl. No.: 11/453,513

(22) Filed: Jun. 15, 2006

(51) Int. Cl. C12M 1/34

(52) U.S. Cl. 435/288.3; 435/288.7

(57) ABSTRACT

A plastic culture insert with a concavity replaces glass coverslip for supporting organic culture. It can be freely placed into and removed from a multi-well plate. The concavity anchors a tissue via collagen polymerization. The concavity is then, after organic culture, sealed by a regular slide for data storage and microscopy imaging analysis.

### FIELD OF THE INVENTION

The present invention relates generally to devices and methods of tissue culture. More specifically, it relates to devices and methods of organic culture and imaging.

### BACKGROUND OF THE INVENTION

Tissue culture is a general term used in modern biotechnology. There are two basic types of tissue cultures, a cell culture and an organic culture. In cell culture, Tissue is dispersed into single cells. These cells form a single-layer in a thickness less than 10 μm after culture. In organic culture, cells stay together in its original structure in a thickness about 200-300 μm.

To set up an organic culture, a piece of isolated tissue, usually in a thickness of 200-300 μm, is embedded onto a culture support via polymerization of a gelling reagent, such as a collagen liquid. The embedded tissue is then immersed under a culture medium and incubated for a few days. During the incubation period, a variety of expensive drugs, hormones, or inhibitors is added into the culture medium to study the response of the tissue. Therefore, smaller wells are meaningful for reducing medium costs.

Organic culture is a difficult and complex process involving numerous factors. Variation of a single parameter can cause unacceptable error or failure.

As a final step, the results of organic cultures are transferred onto slides of microscopy. Problems have been experienced during the development of organic culture technology. Tyndorf et al, in U.S. Pat. No. 5,882,922, teaches a multi-well plate for reducing medium cost in smaller wells. Tyndorf et al has failed to understand the problem of tissue transfer from multi-well plates to slides. Stevens et al, in U.S. Pat. No. 5,605,813, teaches a device for transferring a cell culture into slide. A regular slide of microscopy is built as a culture container with surrounding walls removable. Cultured cells on the regular slide can be sealed by a coverslip. Stevens et al, unfortunately, failed to understand the difference between cell culture and organic culture. The thickness of cell cultured in single layer is less than 10 μm, which can be sealed under flat coverslip. But the thickness of tissue in organic culture is 200-300 μm. Flat coverslip will crash the tissue over a regular slide.

It is a problem to save tissues on slides after organic culture.

Fisher Scientific Company provides a solution for organic culture. A thin glass coverslip is used for supporting tissue in organic culture. Then, the glass coverslip is removed from a multi-well plate to a special slide with a concavity. The concavity provides an extra space for preventing damages to the tissue. Being a step forward in tissue protection, Fisher Scientific Company generates new problems:
1. Glass coverslips, now employed to support organic culture, are very thin and extremely fragile.
2. A pretreatment of the glass coverslip is required for tissue attachment. The quality of the pretreatment declines quickly during storage, which generates significant variation of organic culture.
3. Glass coverslips are flat. Accident detachment of tissue from glass coverslip happens frequently.

Organic culture remains as an unstable and difficult process due to problems of prior arts.

### SUMMARY OF THE PRESENT INVENTION

It is, therefore, an object of the invention to make organic culture easy and reliable.

In the invention, a plastic culture insert with a concavity replaces flat glass coverslip. The concavity protects the tissue throughout organic culture and imaging analysis. Problems of prior arts are solved.

### BRIEF DESCRIPTION OF THE DRAWINGS

FIG. 1 is a top view of a rectangular insert placed in a culture well.

FIG. 2 is an illustrative diagram showing how to use the insert for organic culture.

FIGS. 3a and 3b are side views showing two options of sealing tissue in concavity.

### DETAILED DESCRIPTION OF PREFERRED EMBODIMENT

Regular slides of microscopy have uniform lengths of 75 mm, which is too long for fitting into multi-well plates. Fisher Scientific Company has a concavity built with regular slides so that thin glass coverslips are required to support organic culture in multi-well plates.

The idea of the invention is a combination of a concavity with a small culture insert in shorter than 36 mm for fitting into a multi-well plate.

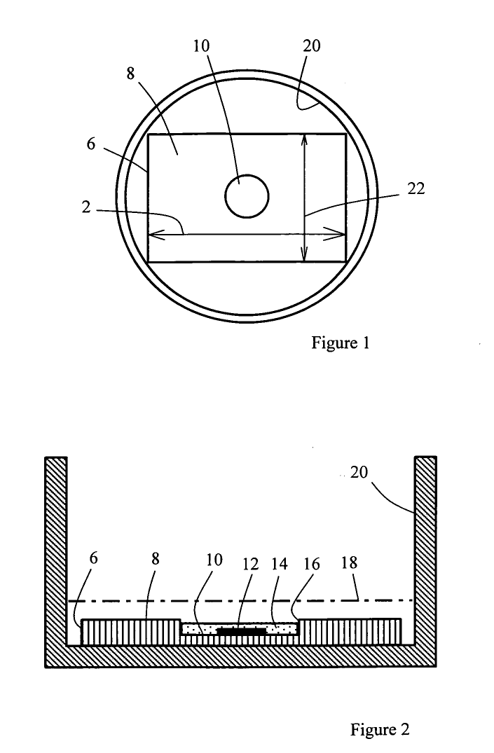

In FIG. 1, an insert 6 is molded with optically clear plastic as a flat piece in a rectangular shape. The thickness of insert 6 is 1.2 mm. A flat surface 8 on insert 6 is used to form seal after organic culture. A width 22 of insert 6 is 24 mm, which is smaller than the width of regular slides. A length 2 of insert 6 should be substantially shorter than the length of regular slides so that insert 6 can be placed into well 20 of a multi-well plate.

FIG. 2 is an illustrative diagram showing insert 6 in well 20 for organic culture. A concavity 10 is located at center of flat surface 8 on top of insert 6. A tissue 12 is embedded in concavity 10 by polymerization of a collagen 14. Concavity 10 is a circle in 8 mm diameter and 0.5 mm depth. The bottom of concavity 10 is 0.5 mm lower than flat surface 8, which keeps tissue 12 away from slides during microscopy imaging analysis.

An advantage of using concavity 10 for organic culture is the enhancement of collagen 14. Collagen is the most popular gelling reagent used in organic culture to embed tissues. Polymerized collagen is a fragile and loose matrix. Detachment of collagen in culture medium has been a common failure in organic culture because the surface of glass coverslip of prior arts is flat and slippery. By using concavity 10, edge 16 anchors collagen 14 tightly and prevents detachment of tissue 12.

During organic culture, a culture medium 18 immerses concavity 10 and maintains growth of tissue 12 for a few days until it is ready for imaging analysis.

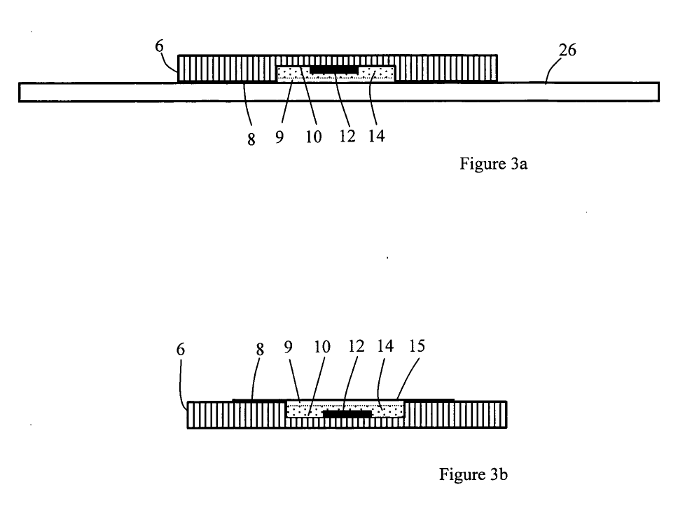

FIGS. 3a and 3b show two options of sealing concavity 10 after culture. In the first option, insert 6 is, like a coverslip, inverted upside down onto a regular slide 26. Tissue 12 is sealed between insert 6 and regular slide 26. A mounting solution 9 is used to remove air under tissue 12, as shown in FIG. 3a.

In the first option, regular slides are used with insert 6. They are dramatically less expensive than specially molded glass concavity slides of prior arts.

FIG. 3b is an alternative option. A thin glass coverslip 15 is placed on top of insert 6 to seal tissue 12. The advantages of this alternative option are:
a. Best quality of imaging through thin glass in microscopy.
b. Easy storage using less space.

In practice, insert 6 can be used in steps as follows:
1. Place multiple units of insert 6 into each well of a multi-well plate, having concavity 10 facing upwards.
2. Transfer tissue 12 to the center of concavity 10.
3. Apply 15 μl of collagen 14 into concavity 10 to embed tissue 12.
4. Wait for polymerization of collagen 14.
5. Add culture medium 18 into well 20 to immerse tissue 12 and collagen 14.
6. Maintain tissue 12 in culture condition for a period of time, usually a few days.
7. Remove insert 6 from multi-well plate.
8. Seal tissue 12 by either a regular slide or a regular coverslip for microscopy imaging analysis.

Although the descriptions above contains specifications, it is apparent to those who skilled in the art that a number of other variations and modifications can be made to the invention without departing from its spirit and scope. Insert 6, for example, can be made with glass. Two units of concavity 10 can be built into insert 6. Rectangular shape of insert 6 can be modified into a disc. Thickness of insert 6 can be less than 1 mm. Insert 6 can be used in a single well culture dish. Therefore, the descriptions as set out above should not be constructed as limiting the scope of the invention but as merely providing illustration of the presently preferred embodiment of the invention.

What is claim is:

1. A device supporting a tissue throughout a process of culture and imaging analysis, comprising;
a body, made with transparent material, being substantially shorter than a regular slide of microscopy, fitting into a culture well, having a flat surface for sealing with said regular slide;
a concavity, built with said body, surrounded by said flat surface, being larger than said tissue, hosting said tissue under a culture medium during said culture, keeping said tissue away from said regular slide during said imaging analysis.

2. The device of claim 1 wherein said insert is made with plastic in a length shorter than 36 mm for fitting into a multi-well plate.

3. A method for supporting a tissue throughout a process of culture and imaging analysis, comprising steps of:
(a) having a device comprising;
a body, made with transparent material, being substantially shorter than a regular slide of microscopy, fitting into a culture well, having a flat surface for sealing with said regular slide;
a concavity, built with said body, surrounded by said flat surface, being larger than said tissue, hosting said tissue under a culture medium during said culture, keeping said tissue away from said regular slide during said imaging analysis.
(b) inserting said device into said well with said concavity accessible from top;
(c) anchoring said tissue in said concavity via a gelling reagent;
(d) incubating said tissue under said culture medium for a period of time;
(e) removing said device from said well;
(f) inverting said device upside down onto said regular slide;
(g) sealing said tissue in said concavity between said device and said regular slide.
